(19) United States
(12) Patent Application Publication
(10) Pub. No.: US 2006/0149119 A1
(43) Pub. Date: Jul. 6, 2006

(54) Method for moderation of sleep disorder

(76) Inventor: Wei-Cheng Wang, Panchiao City (TW)

Correspondence Address: BACON & THOMAS, PLLC, 625 SLATERS LANE, FOURTH FLOOR, ALEXANDRIA, VA, 22314, US

(21) Appl. No.: 11/025,928

(22) Filed: Jan. 3, 2005

(51) Int. Cl. A61N 1/00; A61N 2/00; A61F 7/00

(52) U.S. Cl. 600/9; 607/110; 600/15

(57) ABSTRACT

A method for moderating sleep disorder of a patient includes non-invasively and concurrently electric and magnetic stimulating a group of stimulation areas in a head minimally including vital point baihui DU20 and its vicinity or surrounding area. The electric and magnetic simulation form a mesh like stimulation pattern in the head and has the steps of serially connecting DU20, DU21, DU19 and two GB18 areas for a first polarity electrical stimulation and serially connecting of DU24, two UB4, two SJ20 and two GB6 areas for a second polarity electrical stimulation and has the step of incorporating the same polarity 1 of a permanent magnet placed facing down on DU24, two GB6, DU21, DU19, two GB18, two GB6 and two UB7 areas and the same but opposite to polarity 1 of a permanent magnet placed facing down on two UB4, two SJ20, DU22, DU20 and DU18 areas.

### BACKGROUND OF THE INVENTION

1. Field of the Invention

This invention relates to a method for moderation of sleep disorder.

2. Description of Related Art

Sleep disorder is a chronicle disease affecting hundreds of million people around the world. The causes of sleep disorder are many, including stress, anxiety, psychological factors, lifestyle stressors, environmental interferences, and other physical factors. It is usually treated with such drugs as Hypnotics, Antidepressants and Anxiolytics. Psycotherapy is also employed in certain patients. Other alternative yet common treatment modalities include biofeedback, acupuncture and electric stimulation. For example, there have been electric stimulation devices that apply a small amount of current around both ear lobes which are claimed to have the effect of easing certain types of anxiety. However, other than long term drug therapy, there has been no effective way of long term curing or moderation of sleep disorder.

Wei-Cheng Wang, Head Acupuncture Instrument (HAI), U.S. Pat. No. 6,280,454B1, 2001, describes a head acupuncture device using multiple electrodes integrating magnets and stimulating signal proportional to a rhythm. For applying physiological stimulations to acupuncture points on the head, electrical pulse of 50-110 volts with frequency between 0.3-3.4 KHz has been used.

Further refinement of HAI has led to an important discovery of an effective treatment of sleep disorder by a unique arrangement of electric stimulation pads and permanent magnets inside a HAI. With a wide range of stimulation signals of different strength and frequency, the said device has demonstrated clinically significant effect in treating many types of sleep disorder that otherwise require drug treatment or other forms of psychotherapy.

Other prior arts, such as US Pat. No. 6,418,344 “Method of treating psychiatric disorders by electrical stimulation within the orbital-frontal cerebral cortex”, U.S. Pat. No. 6,591,138 “Implanted control circuit and an array of pads connected to the implanted control circuit”, and U.S. Pat. No. 6,253,109 “System for optimized brain stimulation”, proposed multiple-pad stimulation of a patient's head but these pads are implanted underneath the skull.

Still another prior art, U.S. Pat. No. 5,643,173 “Method of Stress relief”, proposed the treatment of stress disorder by stimulation of two pre-selected acupuncture points on the body with LED type of optical light and concurrent stimulation of the body with low level current using two non-invasive electrodes. These stimulations are applied to vital or acupuncture points below the head.

To overcome the shortcomings, the present invention tends to provide a method for moderation of sleep disorder through an innovative non-invasive electric and magnetic stimulation arrangement.

### SUMMARY OF THE INVENTION

The primary objective of this invention is to treat sleep disorder by electric and magnetic stimulation of a group of selective areas, minimally including the vital area/point Baihui or cortical cortex (DU 20 in FIG. 1), throughout the head of an individual. The method described below further employs a HAI like carrier wherein electronic pads integrating permanent magnets of given magnetic field strengths and polarities are embedded and inter connected. Such a carrier has the effect of facilitating accurate electric and magnetic stimulation of the group of areas throughout the head that have been discovered to have positive effect in moderating certain types of sleep disorder. Either the electrodes and magnets, stimulation generating circuitry, and power supply are all contained and conveniently packaged in such a HAI like carrier, or the stimulation generating and control circuit are in a separate unit connected to the electrodes embedded inside the HAI-like carrier. A user will wear this HAI-like carrier just like wearing a hat or a cap, with the electrodes and permanent magnets in contact with the targeted groups of areas throughout the head during the treatment.

Other objects, advantages and novel features of the invention will become more apparent from the following detailed description when taken in conjunction with the accompanying drawings.

### BRIEF DESCRIPTION OF THE DRAWINGS

FIG. 1 is a perspective view showing one embodiment of a selective group of areas throughout the head that are targets of electric and magnetic stimulation;

FIG. 2 is a schematic view of the electrical interconnection of certain groups of these areas;

FIG. 2A further shows the perspective current distribution amongst the electrodes in FIG. 2;

FIG. 3 is a schematic view of the magnetic polarity intended for each of these areas;

FIG. 3A further shows the perspective magnetic field distribution amongst the permanent magnets shown in FIG. 3;

FIG. 4 is a block diagram of the control circuit inside the controller unit of the present invention;

FIG. 5 is a schematic view of such a HAI-like carrier with the electrodes and magnets embedded inside; and

FIG. 6-8 are alternative embodiments of other groups of selective areas throughout the head that are targets of the electric and magnetic stimulation that provide meaningful comparison of efficacy amongst these different embodiments.

### DETAILED DESCRIPTION OF THE PREFERRED EMBODIMENT

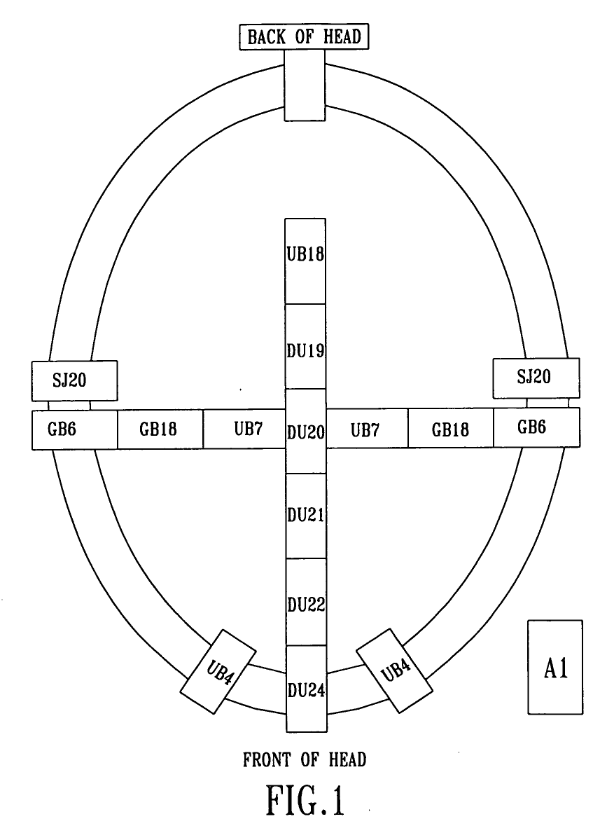

With reference to FIG. 1, there are 16 stimulating areas that are part of this unique arrangement.

DU20, is at the very center on top of the head. (Baihui or cortical cortex)

DU21

DU19

DU22

DU18

DU24

UB4

GB18

UB7

GB6

SJ20

Additionally, there is one area, denoted A1, that is used as a reference area to accurately locate the remaining 16 areas when an HAI-like carrier is being placed on the head of each patient with varying head sizes.

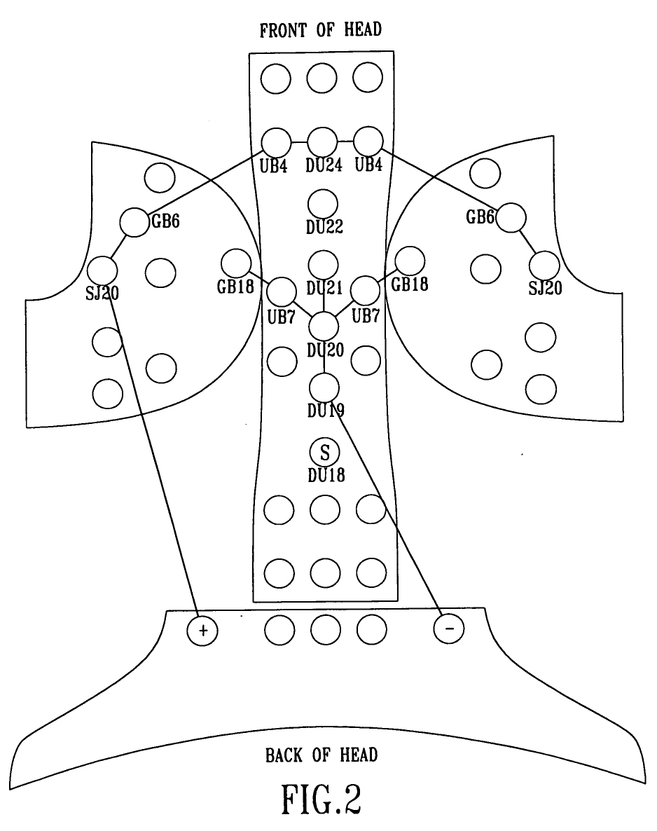

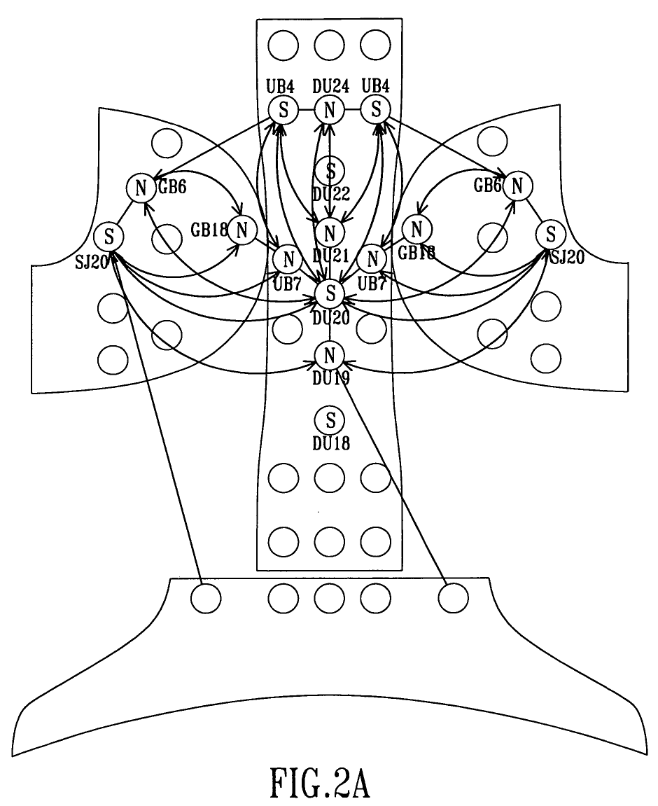

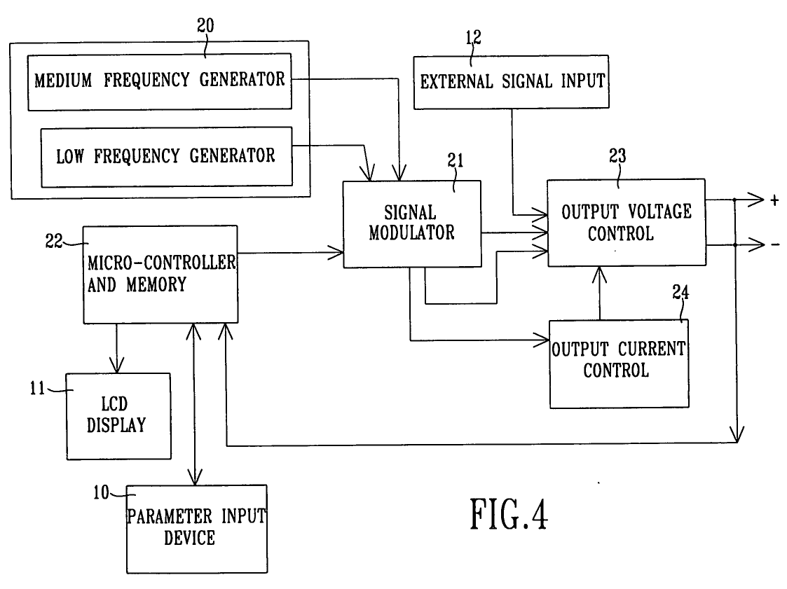

With reference to FIG. 2, DU20, DU21, DU19 and two GB18 areas are all serially connected together to polarity 1 of the controller unit (see FIG. 4). DU24, two UB4, two SJ20 and two GB6 points are serially connected together to polarity 2 of the controller unit.

FIG. 2A depicts a most likely current distribution of electrode arrays shown in FIG. 2. These current flows, with selected strength and frequency, may have varying penetration depths beneath the surface of the head to enhance permeability of neuronal cell membrane at various level that effectively varies the level of local neural activities and to improve intercellular circulation of central never system. In combination, these branches of current flow constitute a mesh like blanket effectively interacting with those portions of brain effecting relaxation. Preferably, the frequency of the electric stimulation signal is a randomized, harmonic or non-harmonic, and alternating pattern, such as music.

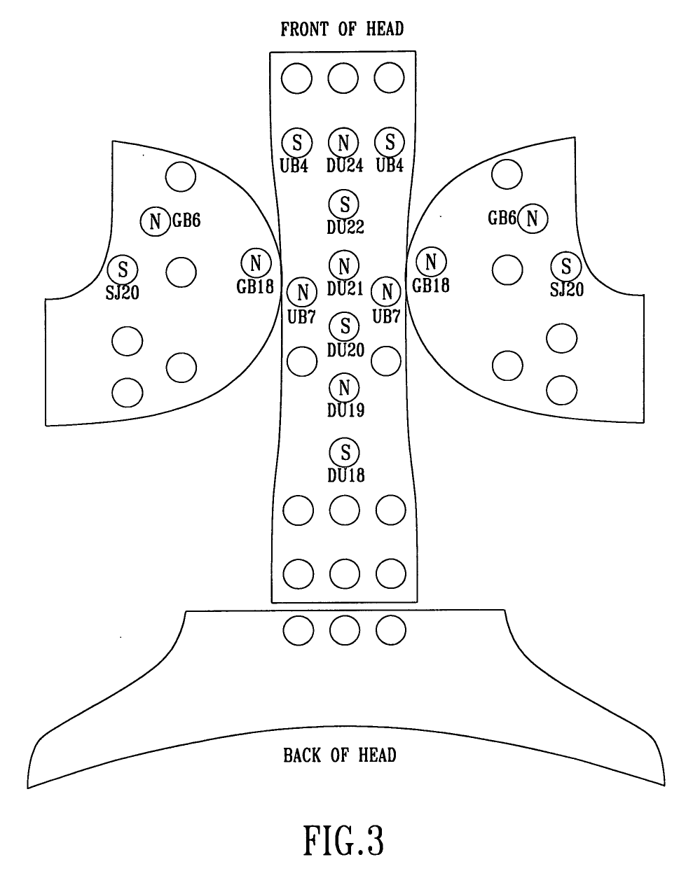

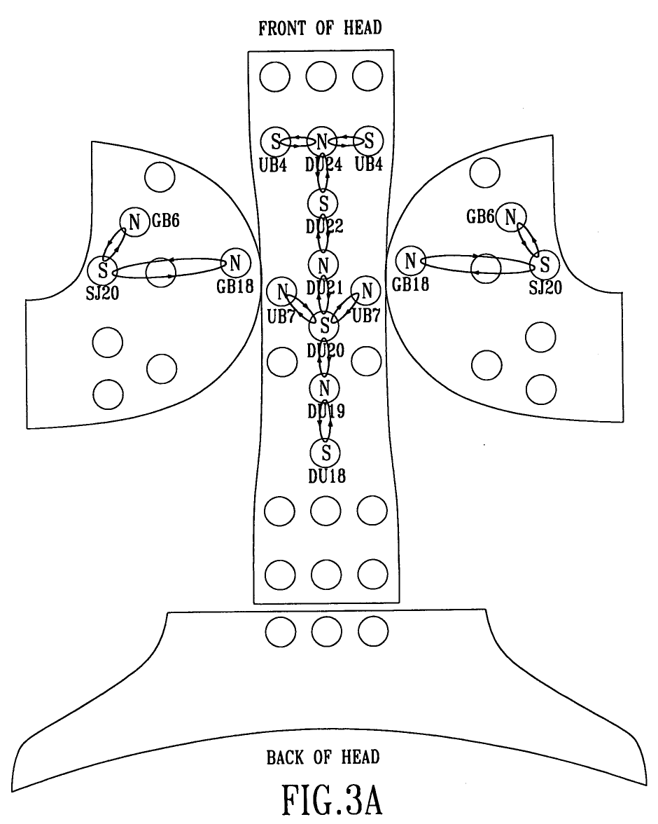

With reference to FIG. 3, north polarity of a permanent magnet is placed facing down on DU24, two GB6, DU21, DU19, two GB18 [symbol] two GB6 and two UB7 areas. South polarity of a permanent magnet is placed facing down on two UB4, two SJ20, DU22, DU20 and DU18 areas.

FIG. 3A depicts a most likely magnetic field distribution of the magnet arrays shown in FIG. 3. These magnet fields, with selected strength for each patient, may have varying penetration depths beneath the surface of the head to effectively maintain a steady gradient of the generated intercellular fluid (ion flow) for a varying level of local activities. In combination, these branches of magnetic fields not only interact with the local area but with the charging ion current flow in FIG. 2A to effect an overall positive reinforcement with those portions of the brain effecting sleep and relaxation.

With reference to FIG. 4, the control circuit of the present invention includes a signal generating unit (20), a signal modulator (21), micro-controller unit (22), output voltage control unit (23) and output current control unit (24).

The signal generating unit (20) generates a low and medium frequency signal and sends the generated signal to the signal modulator (21), which in turn combines the received signal into a desirable final signal according to the instruction of the micro-controller (22). The output voltage control unit (23) receives the output signal from the signal modulator (21) and the external signal input (12) such as music and controls, according to the instruction of the micro-controller controlled output current control unit (24), the strength of the stimulation signal. The two-polarity output of the output voltage control unit (23) is then connected to the two electric inputs of the carrier such as a HAI.

The micro-controller unit (22) is capable of displaying information on a display (11) such as an LCD (Liquid Crystal Display) and receives external input from an input device (10) such as a keyboard.

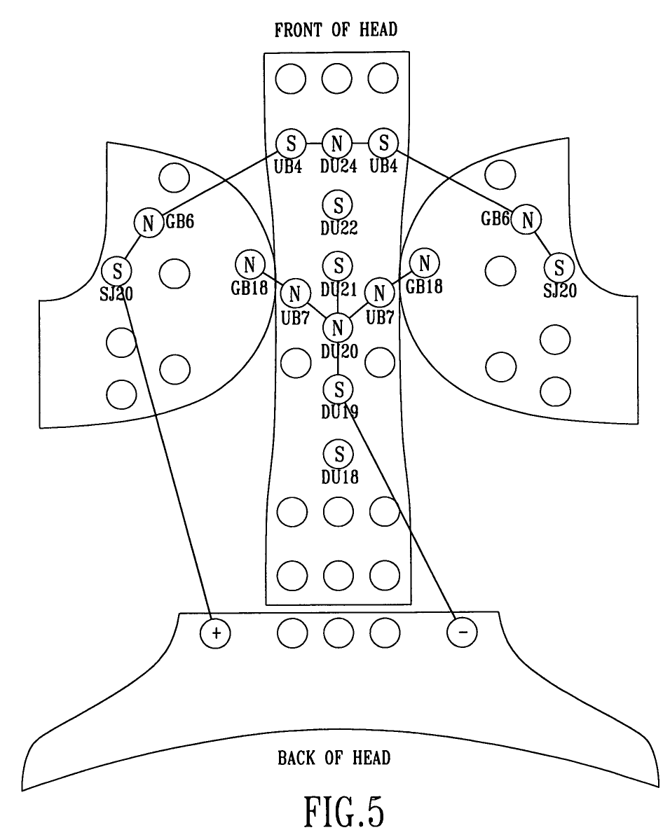

FIG. 5 shows a preferred embodiment of such a HAI-like carrier in accordance with the present invention, including a stretchable cover inside which are housed the inter connected electrodes and magnets.

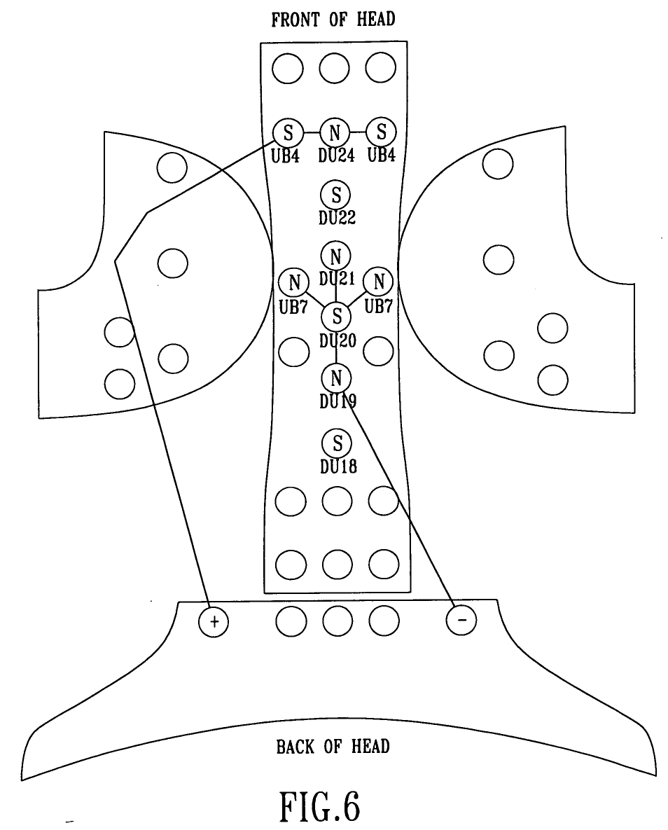

FIG. 6 depicts another group of selective areas throughout the head that were also tested to compare with the results of the preferred embodiment of FIG. 5. It is the purpose of this embodiment to decide the difference in efficacy between two different types of connections both of which include DU20 as part of the stimulation point.

Two groups of test subjects with similar sleep disorder symptoms were treated separately with these two different embodiments. The limited clinical trials first revealed that the difference in efficacy for sleep disorder moderation between these two embodiments is not very significant.

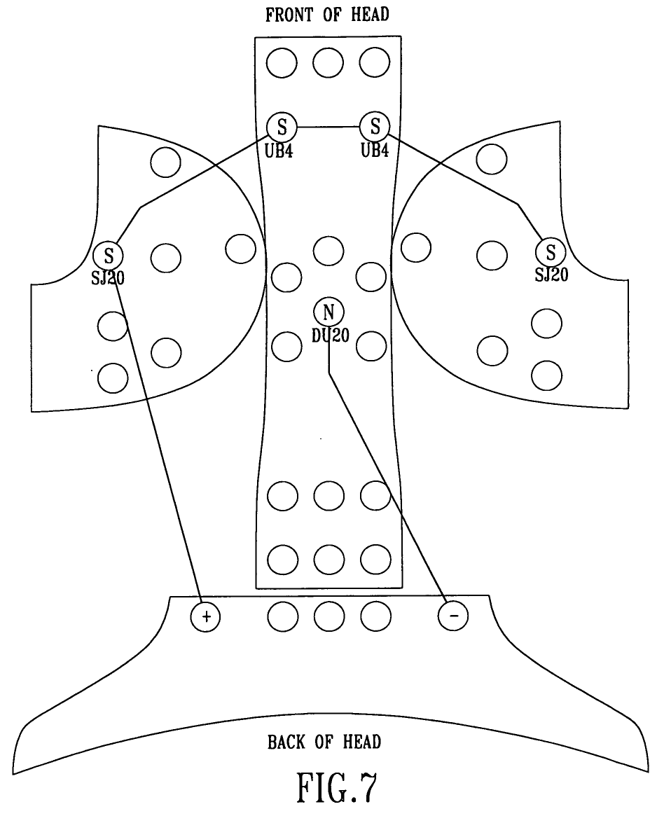

FIG. 7 represents yet another embodiment of the present invention, which includes only the area/point Baihui or cortical cortex (DU20) connected to electrical polarity 1 (negative) and with the north polarity of a permanent magnet placed facing down on it, and two SJ20 and two UB4 serially connected to the electrical polarity 2 (positive) and with the south polarity of each permanent magnet placed facing down on each of these locations. Based on our many trials, it is postulated that the area/point Baihui or cortical cortex (DU20) seems to play the most important role in the effective moderation of the sleep disorder symptom. This embodiment includes a minimal group of only one pair of areas on the head, DU20 as one area and SJ20 and UB4 together as the other area. Treatments of test subjects using this and the preferred embodiment of FIG. 5 revealed that the treatment time to effect similar results is about 10% longer using the embodiment of FIG. 7 than that using the embodiment of FIG. 5. However, the embodiment of FIG. 7 is still considered sufficiently effective for the moderation of the sleep disorder under the same clinical criteria used in our trials.

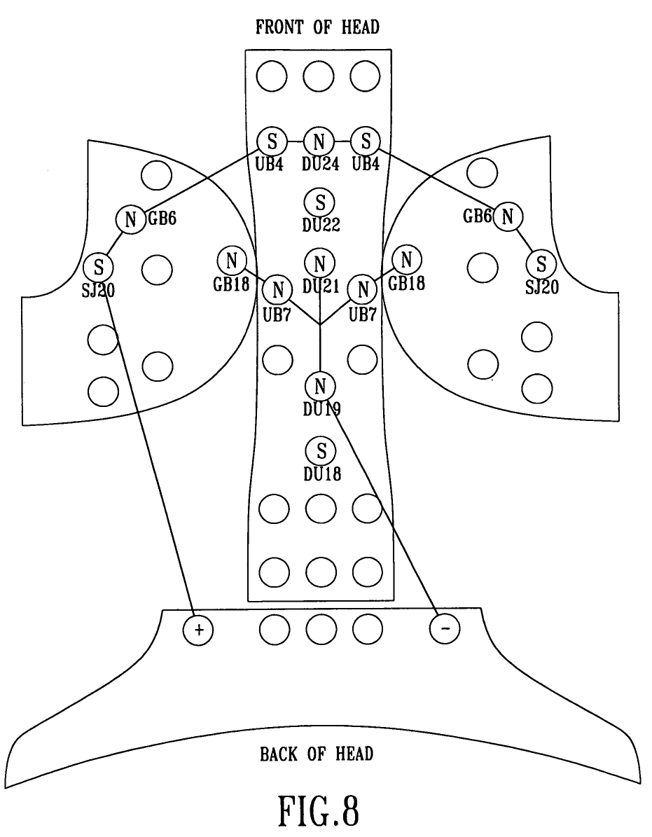

FIG. 8 represents another embodiment that is the same as that described in FIG. 5 minus the area/point Baihui or cortical cortex (DU20). The clinical test result using this embodiment shows a significant reduction of efficacy in the treatment of the sleep disorders compared to those using the embodiments of FIG. 5, FIG. 6, and FIG. 7. It is considered ineffective in the treatment of the sleep disorder.

Our discovery derives from findings from years of EEG data and the oriental acupuncture theorem regarding the potential biological effects of electromagnetic stimulation of the particular group of vital points throughout the head.

Volunteer patients were tested for sleep disorder. All subjects use an HAI-like carrier on their head for approximately 30 minutes for each treatment session. Different sizes of the HAI-like carrier were used for each tested subject to ensure that the electrodes and magnets inside the carrier are placed directly on the group of targeted vital points throughout the head. During each treatment, the stimulating waveform and magnitude level of each tested subject were recorded. In order to clinically validate the trials, methods used to record the stimulated strength level are conformed to clinically acceptable ones including the stimulated electrical current. The total treatment consists of three treatment sessions each week for four weeks. After four weeks, the extent of the reduction in sleep disorder was observed and recorded for each test group. In most of cases, sleep disorder was reduced significantly. To ensure that the reduction was not temporary, each patient was required to report to the physician once every week, after the treatment session was completed, the status of his/her sleep patterns. The majority of the tested subjects who were determined to have a significant reduction of sleep disorder continued to report either no recurring of the sleep disorder or a sustained improvement of the conditions over what it was before the treatment.

The area of each electrode pad containing also a magnet used in these applications tends to be small (19 mm in diameter for the ones used in various embodiments described in FIGS. 2 to 8), in order to provide a local stimulation to a functional group of cortical cortex and inner cerebrum (cerebral cortex). The mechanism of action is not thoroughly understood. However, we expect that the sleep disorder conditions should be moderated due to the combination effects of the electrical and magnetic stimulation that will be enhancing cellular permeability and improving intercellular fluid circulation. The technique accomplished by the device is referred to as electro-acupuncture or non-invasive nerve stimulation, and has components of acupuncture effect and acupressure. While we used the HAI-like non-invasive stimulation carrier, any suitable means of applying the electromagnetic stimulation to the group of areas on the head median nerve should work to moderate sleep disorder.

While the preferred embodiments of the methods have been described in reference to the environment in which they were developed, they are merely illustrative of the principles of the inventions. Other embodiments and configurations may be devised without departing from the spirit of the inventions and the scope of the appended claims.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A method for moderating sleep disorder of a patient comprising non-invasively and concurrently electric stimulating a group of stimulation areas in a head minimally including DU20 located at a center of the head and its vicinity or surrounding area.

2. The method as claimed in claim 1, wherein frequency of the electric stimulation is a randomized, alternate pattern.

3. The method as claimed in claim 1, wherein the electric simulation forms a mesh like stimulation pattern in the head.

4. The method as in claim 2, wherein the randomized, alternate pattern is a music rhythm.

5. The method as claimed in claim 1, wherein the non-invasively electric stimulation comprises serially connecting DU20, DU21, DU19 and two GB18 areas for a first polarity electric stimulation and serially connecting of DU24, two UB4, two SJ20 and two GB6 areas for a second polarity electric stimulation.

6. The method as claimed in claim 3, wherein the non-invasively electrical stimulation comprises serially connecting DU20, DU21, DU19 and two GB18 areas for a first polarity electric stimulation and serially connecting of DU24, two UB4, two SJ20 and two GB6 areas for a second polarity electric stimulation.

7. The method as claimed in claim 4, wherein the first polarity electric stimulation is opposite to that of the second polarity electric stimulation.

8. A method for moderating sleep disorder of a patient comprising non-invasively and concurrently magnetic stimulating a group of stimulation areas in a head minimally including DU20 and its vicinity or surrounding area.

9. The method as claimed in claim 8, wherein the magnetic simulation form a mesh like stimulation pattern in the head.

10. The method as claimed in claim 8, wherein the non-invasively magnetic stimulation comprises using a polarity the same as that of a permanent magnet placed facing down on DU24, two GB6, DU21, DU19, two GB18, two GB6 and two UB7 areas and the same but opposite to the polarity of a permanent magnet placed facing down on two UB4, two SJ20, DU22, DU20 and DU18 areas.

11. A method for moderating sleep disorder of a patient comprising non-invasively and concurrently electric and magnetic stimulating a group of stimulation areas in a head minimally including DU20 located at a center of the head and its vicinity or surrounding area.

12. The method as claimed in claim 11, wherein frequency of the electric stimulation is a randomized, alternate pattern.

13. The method as claimed in claim 11, wherein the electric and magnetic simulation form a mesh like electrical and magnetic stimulation pattern in the head.

14. The method as in claim 12, wherein the randomized, alternate pattern is a music rhythm.

15. The method as claimed in claim 11, wherein the non-invasively electric and magnetic stimulation comprises serially connecting DU20, DU21, DU19 and two GB18 areas for a first polarity electric stimulation and serially connecting of DU24, two UB4, two SJ20 and two GB6 areas for a second polarity electric stimulation.

16. The method as claimed in claim 13, wherein the non-invasively magnetic stimulation comprises using a polarity the same as that of a permanent magnet placed facing down on DU24, two GB6, DU21, DU19, two GB18, two GB6 and two UB7 areas and the same but opposite to the polarity of a permanent magnet placed facing down on two UB4, two SJ20, DU22, DU20 and DU18 areas.

17. The method as claimed in claim 14, wherein the first polarity electric stimulation is opposite to that of the second polarity electric stimulation.
